(19) United States
(12) Patent Application Publication
(10) Pub. No.: US 2006/0239396 A1
(43) Pub. Date: Oct. 26, 2006

(54) Method and tomography apparatus for fast volume scanning of an examination region

(76) Inventors: Herbert Bruder, Hochstadt (DE); Martin Petersilka, Adelsdorf (DE)

Correspondence Address: SCHIFF HARDIN, LLP;PATENT DEPARTMENT, 6600 SEARS TOWER, CHICAGO, IL, 60606-6473, US

(21) Appl. No.: 11/393,034

(22) Filed: Mar. 30, 2006

(30) Foreign Application Priority Data
Mar. 30, 2005 (DE) 10 2005 014 853.0

(51) Int. Cl. H05G 1/60; A61B 6/00; G01N 23/00; G21K 1/12

(52) U.S. Cl. 378/4

(57) ABSTRACT

In a method and a tomography apparatus for fast volume scanning of an examination region with at least two acquisition systems. Given a helical scanning of the examination region, a particularly fast scanning of the volume is achieved by projection gaps of the examination region in projections acquired with the first acquisition system being filled by projections with the second acquisition system.

### BACKGROUND OF THE INVENTION

1. Field of the Invention

The invention concerns a tomography apparatus for fast volume scanning of an examination region with at least two acquisition systems that each acquire projections of the examination region. The invention moreover concerns a method for fast volume scanning of the examination region with such a tomography apparatus.

2. Description of the Prior Art

Tomography apparatuses with two acquisition systems are known, for example, from U.S. Pat. No. 4,991,190, DE 29 51 222 A1 and DE 103 02 565 A1. The advantage of such tomography apparatuses relative to an apparatus with only one acquisition system is in the ability to examine a subject with an increased scan speed or with an increased scan resolution.

High scan speeds are advantageous when movement artifacts should be minimized in the reconstructed image. A high scan speed ensures that all projections used for reconstruction of an image, which projections are from various rotation angle positions, acquire the same movement state of a subject (for example the same cardiac phase of a heart) to the greatest possible extent. In the known tomography apparatuses, both acquisition systems are arranged around a common rotation axis and are arranged offset relative to one another by an angle of 90° in the rotation direction, such that the scan speed can be doubled with the use of suitable reconstruction methods.

Tomography apparatuses with a number of acquisition systems also can be used for generation of images with a higher resolution. For this purpose the acquisition systems are arranged around the common rotation axis such that the projections of both acquisition systems for the same projection direction exhibit an offset relative to one another that is smaller than a detector element. A higher-resolution image can be calculated by evaluation of the projections acquired by both acquisition systems in succession from the respective projection directions. A higher resolution is advantageous, for example, in the examination of blood vessels, in which small examination volumes must be scanned.

For reconstruction of an image, the projections generated by both acquisition systems are registered with respect to one another both in the operating mode for increasing the scan speed and in the operating mode for increasing the scan resolution. The registration of the data ensues with knowledge of the system angle at which the acquisition systems are arranged in the azimuthal direction around a common rotation axis.

### SUMMARY OF THE INVENTION

An object of the invention is to extend the functionality of a tomography apparatus with multiple acquisition systems.

According to the invention, a tomography apparatus has at least two acquisition systems that respectively acquire projections of (projection data from) an examination region. In a spiral mode (also known as a helical scan operation) of the tomography apparatus, a rotation of both acquisition systems around a common rotation axis is implemented together with displacement of the examination region relative to both acquisition systems and together with displacement of the acquisition systems relative to one another, such that projection gaps of the examination region in projections acquired with the first acquisition system can be completed filled by projections acquired with the second acquisition system.

The filling of the projection gaps succeeds when both acquisition systems are offset relative to one another by a fixed set angle in the rotation direction. Two projections thus are acquired at each projection direction at various points in time in each rotation. Due to the feed of the examination region in the direction of the rotation axis (which feed occurs simultaneously in a spiral scan), the projections for the same projection direction are acquired at different positions along the rotation axis, such that the projections of the second acquisition system acquire other partial regions that are not covered by the projections of the first acquisition system. Projection gaps that arise due to a high feed speed in spiral scanning with only one acquisition system thus can be filled by the projections of the second acquisition system.

In the helical scanning with a computed tomography apparatus, the pitch value specifies the ratio between the feed of the table plate or the examination region per gantry rotation and the slice thickness of the detector. Given a helical scan of an examination region of, for example, 250 mm diameter with only one acquisition system, whereby the conical angle of the x-rays is set to approximately 25 degrees for exposure of a 32×0.6 mm line detector, a maximal pitch value of 1.7 can be provided without projection gaps occurring within the examination region. In contrast to this, in an inventive computer tomograph with two acquisition systems offset relative to one another by 90° in the azimuthal direction (i.e. an angle of 90° having the rotation axis proceeding through its vertex) it is possible to implement the scan with a pitch value of 3 (higher by approximately a factor of 1.75) without the occurrence of projection gaps.

Give the use of a single acquisition system, such a fast feed of the examination region in which no projection gaps arise could be achieved only when the detector exhibits a correspondingly large dimension in the direction of the rotation axis and when the x-ray beam generated by an x-ray radiator possesses a correspondingly large conical angle.

The design of detectors with a large dimension in the direction of the rotation axis is, however, complicated, and the expansion of the x-ray beam leads to a high thermal load of the x-ray radiator that arises due to a significant inclination of the anode plate relative to the electron beam. Moreover, the complexity of the algorithms for calculation of a reconstructed image is increased due to the usage of detectors that acquire a number of detector lines given each projection.

The invention thus represents an efficient alternative to tomography apparatuses with only one acquisition system, and is designed specifically for a fast volume scanning, but the overall functionality of a tomography apparatus with at least two acquisition systems is also expanded by the invention. In addition to the already-known operating modes for scanning of an examination region with a high scan speed that enables a reduction of movement artifacts and with a high scan resolution that ensures an acquisition of particularly small scan volumes, the tomography apparatus can inventively be operated in a further operating mode with which a particularly fast volume scan is possible.

In principle, the arrangement of both acquisition systems relative to one another in the direction of the rotation axis is not significant for the inventive tomography apparatus for fast volume scanning. With regard to an optimally broad field of use of the tomography apparatus, however, it is advantageous when both acquisition systems are arranged in one measurement plane, thus without an offset in the direction of the rotation axis. In this case not just one fast volume scan can be implemented by means of a spiral scan. Scans with a high scan speed additionally can be realized in the manner described above, in which the same scan volume must be acquired in the direction of the rotation axis by both acquisition systems.

The acquisition systems are arranged offset by 90° relative to one another in the rotation direction. The temporal difference between successive projections that are acquired for one projection plane is maximum due to the offset of the two acquisition systems by 90°, such that the partial region (which can serve for filling the projection gaps) acquired by the projection of the second acquisition system is also maximum given helical scanning with the feed of the examination region in the direction of the rotation axis.

As is the case, for example, in a computed tomography apparatus, each acquisition system has a radiator for generation of radiation and a detector for acquisition of the projections. Each detector preferably is composed of a number of detector elements in columns and rows, each detector element generating an attenuation value dependent on the attenuation by the examination subject of the radiation incident thereon.

In an embodiment of the invention, both acquisition systems exhibit equally large measurement regions. It is possible for the measurement regions to be dimensioned with different sizes. In this case, the fast volume scanning is preferably implemented on the basis of the respective partial regions of the projections of both acquisition systems that cover an optimally large common measurement region. Alternatively, a measurement region can be used for scanning of an examination region that is larger than the smaller measurement region of both acquisition systems. In this case, a reconstruction of the image dependent on the partial region of the measurement region occurs. An inner partial region of the measurement region that corresponds to the largest common measurement region of both acquisition systems is covered without gaps by projections of both acquisition systems, such that a reconstruction is possible without losses in the resolution of the image in the direction of the rotation axis. By contrast, the outer measurement region exhibits projection gaps that are not covered by the second acquisition system due to the smaller measurement field. In this partial region, a reconstruction is only possible only with a loss of resolution in the direction of the rotation axis by the missing information being replaced with or interpolated from adjacently-arranged projections.

Ideally, the measurement regions of both acquisition systems are executed such that they can displaced so that an adjustment adapted to the examination condition can ensue.

The reconstruction of an image is advantageously implemented on the basis of the acquired projections of both acquisition systems, the displacement position in the direction of the rotation axis and the angle position in the rotation direction of the acquisition system being detected and further processed for unambiguous geometric association of each projection.

### DESCRIPTION OF THE DRAWINGS

FIG. 1 shows an inventive tomography apparatus for fast volume scanning of an examination region with two acquisition systems in a perspective view.

FIG. 2 shows both acquisition systems in cross-section.

FIG. 3 illustrates projections of both acquisition systems in a projection plane for gapless scanning of the examination region.

### DESCRIPTION OF THE PREFERRED EMBODIMENTS

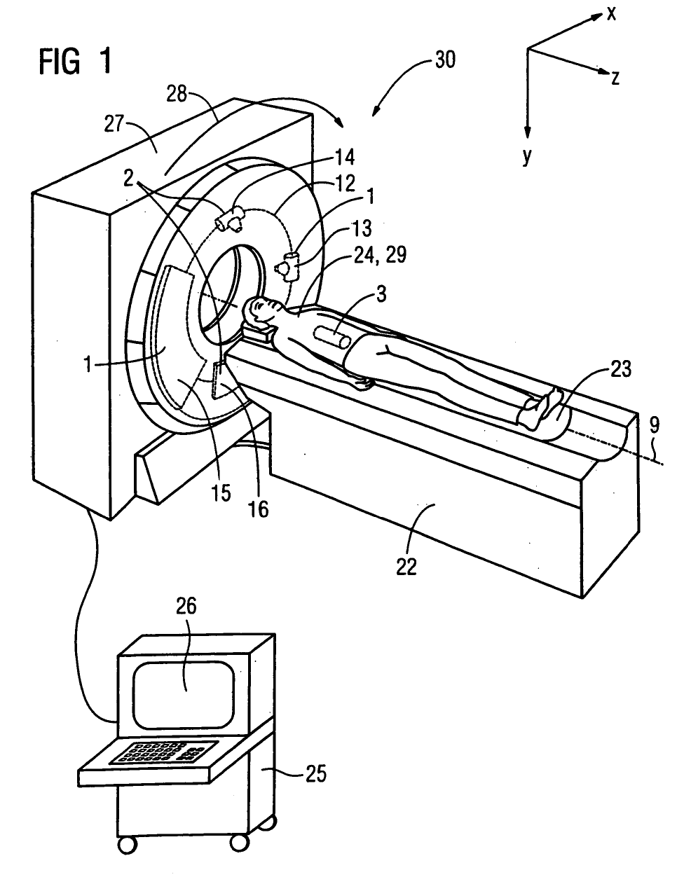

An inventive tomography apparatus (here a computed tomography apparatus 30) is shown in perspective view in FIG. 1.

Two acquisition systems 1, 2 (mounted such that they can rotate on a gantry) for acquisition of projections 4, 5, 6, 7, 8 of an examination region 3 from a number of different projection directions 31, 32 are located inside the computed tomography apparatus 30. The second acquisition system 2 exhibits an angular offset of 90° in the shown rotation direction 28 relative to the first acquisition system 1. Both acquisition systems 1, 2 are essentially located in the same measurement plane 12.

A bearing device 22 with a movable table plate 23 on which a subject 24 (for example a patient 29) can be borne is associated with the computed tomography apparatus 30. The table plate 23 can be displaced in the direction of the rotation axis 9, such that an examination region 3 (connected with the subject 24) can be moved through an opening in the housing 27 of the computer tomograph into the measurement regions 18, 19 of both acquisition systems 1, 2. The examination region 3 of the subject 24 and both acquisition systems 1, 2 can be displaced relative to one another in the direction of the rotation axis 9 in this manner.

To acquire projections 4, 5, 6, 7, 8, the acquisition systems 1, 2 respectively have radiators 13, 14 in the form of an x-ray tube and detectors 15, 16 arranged opposite this. Each detector 15; 16 is composed of a number of detector elements 17 strung in columns and rows. Each radiator 13, 14 generates a ray in the form of a fan-shaped x-ray beam which penetrates the respective measurement region 18, 19 of the acquisition system 1, 2. The x-ray radiation subsequently strikes the detector elements 17 of the respective detectors 15, 16. The detector elements 17 generate an attenuation value dependent on the attenuation of the x-ray radiation passing through the respective measurement region 18, 19 of the acquisition system 1, 2. For example, the conversion of the x-ray radiation into an attenuation value respectively ensues by means of a photodiode optically coupled with a scintillator or by means of a directly-converting semiconductor. In this manner, each detector 15, 16 generates a set of attenuation values which are acquired for a specific projection direction 31, 32 of the acquisition system 1, 2.

Due to rotation of the gantry with simultaneous, continuous feed of the examination region 3 in the direction of the rotation axis 9, projections 4, 5, 6, 7, 8 are acquired from a number of different projection directions 31, 32 at various positions 33, 34, 35, 36 along the rotation axis 9, or along the examination region 3. The projections 4, 5, 6, 7, 8 of both acquisition systems 1, 2 acquired in this manner are transmitted to a computer 25 and calculated into an image. Before the actual reconstruction of the image, according to a known interpolation method, those projections that would be acquired without displacement of the examination region 3 (thus given exclusive rotation of the acquisition systems 1, 2) in the projection planes perpendicular to the measurement plane 12 of the computed tomography apparatus 30 are interpolated from projections of the spiral scan that are adjacent in the direction of the rotation axis. According to the typical method such a pre-processing is possible without loss of image resolution only when no projection gaps 10, 11 of the examination region 3 occur in the scanning of the examination region 3. Such projection gaps 10, 11 are prevented according to the inventive tomography apparatus and according to the inventive method, such that the known pre-processing methods can be used for reconstruction of the image. The reconstructed image can be visually displayed to operating personnel on a display unit 26.

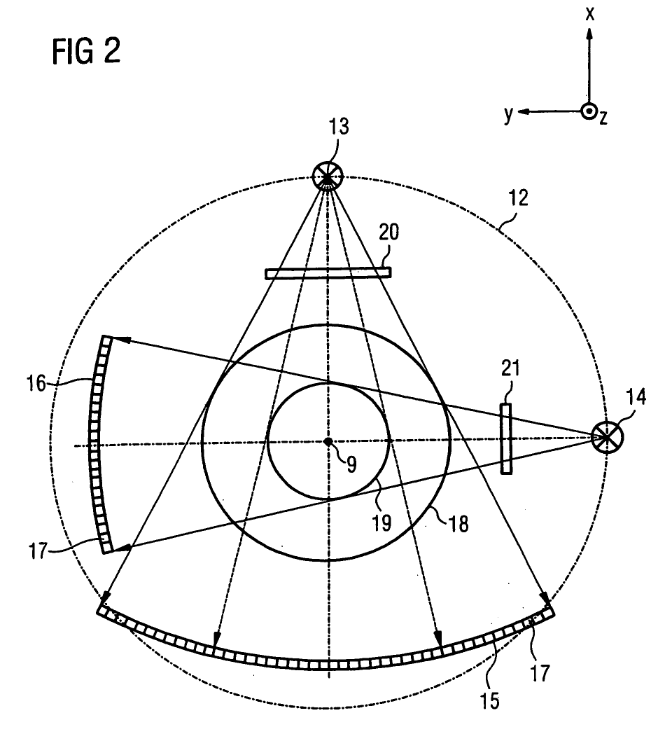

FIG. 2 shows both acquisition systems 1, 2 in detail. One line of the detectors 15, 16 with a number of detector elements 17 is shown in cross-section, with only one of the detector elements 17 being provided with a reference character. The respective measurement fields of the detectors 15, 16 are of different sizes, such that detectable x-ray beams of different sizes result for both acquisition systems 1, 2. The maximum fan aperture angle of a detectable x-ray is, for example, 55° in the first acquisition system 1 and 25 degrees in the second acquisition system 2. Due to the rotary scanning of the examination region 3, a maximum measurement region of 500 mm in diameter results for the first acquisition system 1 and a maximum measurement region of 250 mm in diameter results for the second acquisition system 2. The first measurement region 18 typically serves for scanning of the entire body cross-section of a patient while the second measurement region 19 is used for scanning of a heart region of the patient.

Adjustable diaphragms 20, 21 are respectively associated with the acquisition systems 1, 2. The diaphragms 20, 21 allow the x-ray radiation of the respective acquisition systems 1, 2 to be set to arbitrary measurement regions. For the following example, the fan aperture angle is selected by a diaphragm setting such that both acquisition systems 1, 2 as shown cover the same measurement region 19 of 250 mm diameter. Moreover, the conical angle is adjusted by a suitable diaphragm setting such that 32×0.6 mm lines of the respective detectors 1, 2 are exposed. Both measurement regions thus exhibit the same dimension both in the rotation axis direction and in the azimuthal direction.

Given use of a single acquisition system 1 with such a set measurement region 19 given helical scanning of an examination region 3, the computed tomography apparatus 30 can be operated with a maximum pitch value of 1.7 without projection gaps arising in the examination region 3 to be scanned. The pitch value is calculated from the relationship ratio between the table feed or the feed of the examination region 3 per gantry rotation in the direction of the rotation axis 9 and the slice thickness that is acquired of the measurement region 19 by the detector 15.

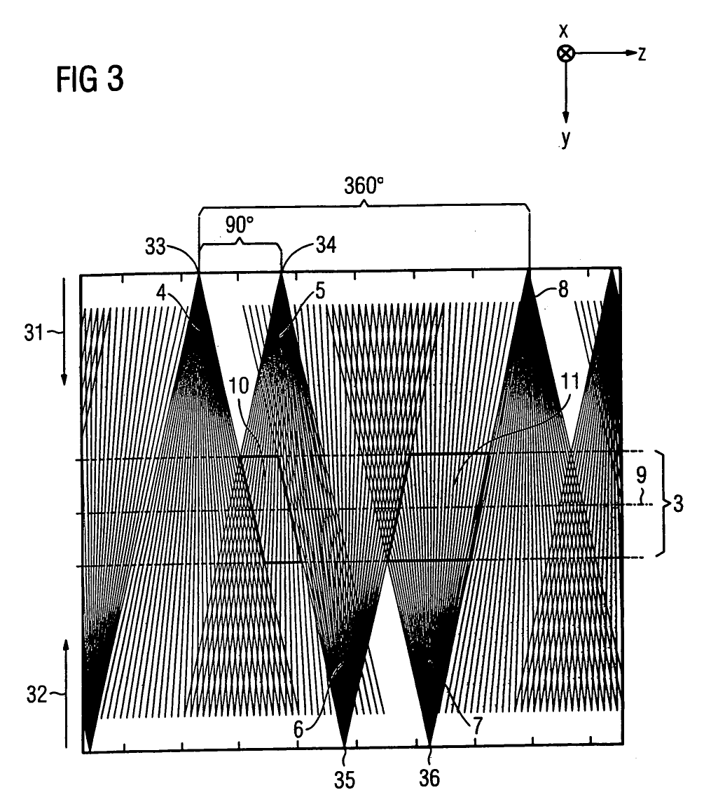

In the inventive computed tomography apparatus, the maximum predeterminable pitch value can be significantly increased by projection gaps 10, 11 of the examination region 3 (as shown in FIG. 2) given projections 4, 6, 8 acquired with the first acquisition system 1 being filled by projections 5, 7 with the second acquisition system 2. FIG. 3 shows in detail projections 4, 5, 6, 7, 8 of both acquisition systems 1, 2 that are acquired during a spiral scan in the same projection plane 12 from two projection directions 31, 32 offset from one another by 180°.

For each revolution of the two acquisition systems 1, 2, two projections 4, 5 and 6, 7 are acquired at each projection direction 31, 32. Due to the offset of both acquisition systems 1, 2 in the rotation direction 28, both acquisition systems 1, 2 do not pass through the same projection direction 31, 32 at the same time, but rather at varying points in time 33, 34 and 35, 36. The projections 4, 5 and 6, 7 are for this reason not acquired at the same position, but rather at varying positions 33, 34 and 35, 36 in the direction of the rotation axis 9 dependent on the feed of the examination region. Projection gaps 10, 11 that arise in the scanning with only the first acquisition system 1 given the high adjusted pitch value are filled by the projections 5, 7 of the second acquisition system 2 in each revolution.

Given the predetermined measurement region 19 of 250 mm diameter, pitch values of maximally 3 can be set in this manner without projection gaps 10, 11 occurring within the examination region 3. In this example the volume scanning thus can be implemented faster by a factor of 1.75 in comparison with a computed tomography apparatus with only one such acquisition system. Such a fast volume scanning is possible with only one acquisition system when the detector is formed from 56×0.6 mm lines instead of the 32×0.6 mm lines. The design of such detectors, however, is possible only with an additional expense. Moreover, the complexity of the reconstruction algorithms that must be used for calculation of image information from the projections increases due to the expanded ray geometry. The inventive tomography apparatus thus offers an efficient possibility for fast volume scanning of an examination region.

The maximum predeterminable pitch value (and thus the advantage relative to the scanning with only one acquisition system) is dependent on a series of different factors. Significant influencing factors are the offset of the two acquisition systems relative to one another in the azimuthal direction, the dimension of the measurement region in the scan plane (which is necessary for scanning of the examination region) and the geometric arrangement of the detectors and the radiator.

A fast volume scanning also is possible when the radiator radiates a ray other than a fan-shaped ray. It is thus possible to implement a fast volume scan with two acquisition systems arranged such that they can rotate around a common rotation axis, in which acquisition systems the radiators generate a diverging or parallel ray beam.

For reconstruction of an image, for example of a slice or volume image, the projections 4, 5, 6, 7, 8 of both acquisition systems 1, 2 are registered with respect to one another. For this purpose, the acquired projections 4, 5, 6, 7, 8 are transmitted, for example, to the computer 25 or to a separately provided reconstruction unit. An image can be calculated using a known reconstruction algorithm with knowledge of the rotation angle position and of the acquisition position in the direction of the rotation axis 9.

Although modifications and changes may be suggested by those skilled in the art, it is the intention of the inventors to embody within the patent warranted hereon all changes and modifications as reasonably and properly come within the scope of their contribution to the art.

We claim as our invention:

1. A tomography apparatus for fast volume scanning of an examination region, comprising:
at least two projection data acquisition systems that each acquire projections, comprising projection data, from an examination region; and
said at least two acquisition systems being mounted and controlled to rotate around a common rotation axis proceeding through said examination region with a relative displacement between said examination region and said at least two acquisition systems along said rotation axis, with said at least two acquisition systems being disposed relative to each other, to cause projection gaps of said examination region in projections acquired with a first of said at least two acquisition systems to be filled by projections acquired with a second of said at least two acquisition systems.

2. A tomography apparatus as claimed in claim 1 wherein said at least two acquisition systems are disposed in a common measurement plane.

3. A tomography apparatus as claimed in claim 1 wherein said first of said at least two acquisition systems is offset from said second of said at least two acquisition systems by an angle of 900 in an azimuthal direction.

4. A tomography apparatus as claimed in claim 1 wherein each of said at least two projection data acquisition systems comprises a radiator that emits radiation into said examination region and a detector that detects said radiation after passing through said examination region, with the detected radiation forming the projection data for a projection.

5. A tomography apparatus as claimed in claim 4 wherein each detector comprises a plurality of detector elements disposed in columns and rows, each detector element generating an attenuation value dependent on said radiation incident thereon.

6. A tomography apparatus as claimed in claim 4 wherein each detector has a measurement region sensitive to said radiation, and wherein the respective measurement regions are equal in size.

7. A tomography apparatus as claimed in claim 6 wherein the respective measurement regions of said at least two projection data acquisition systems are displaceable.

8. A tomography apparatus as claimed in claim 1 comprising an image reconstruction computer that reconstructs an image of said examination region from the projections acquired by said at least two acquisition systems.

9. A tomography apparatus as claimed in claim 1 comprising a computed tomography scanner in which said at least two projection data acquisition systems are mounted for rotation around said examination region, and comprising an image reconstruction computer programmed to implement a computed tomography image reconstruction algorithm using the projections acquired by said at least two projection data acquisition systems, to generate a computed tomography image of said examination region.

10. A tomography method for fast volume scanning of an examination region, comprising the steps of:
acquiring projections, comprising projection data, from an examination region from each of at least two data acquisition systems; and
mounting and controlling said at least two acquisition systems being mounted and controlled to rotate around a common rotation axis proceeding through said examination region with a relative displacement between said examination region and said at least two acquisition systems along said rotation axis, with said at least two acquisition systems being disposed relative to each other, and causing projection gaps of said examination region in projections acquired with a first of said at least two acquisition systems to be filled by projections acquired with a second of said at least two acquisition systems.

11. A tomography method as claimed in claim 10 comprising disposing said at least two acquisition systems in a common measurement plane.

12. A tomography method as claimed in claim 10 comprising offsetting said first of said at least two acquisition systems from said second of said at least two acquisition systems by an angle of 90° in an azimuthal direction.

13. A tomography method as claimed in claim 10 wherein each of said at least two projection data acquisition systems comprises a radiator that emits radiation into said examination region and a detector that detects said radiation after passing through said examination region, and forming the projection data for a projection from the detected radiation.

14. A tomography method as claimed in claim 13 comprising forming each detector of a plurality of detector elements disposed in columns and rows, and from each detector element, generating an attenuation value dependent on said radiation incident thereon.

15. A tomography method as claimed in claim 13 wherein each detector has a measurement region sensitive to said radiation, and making the respective measurement regions equal in size.

16. A tomography method as claimed in claim 16 comprising displacing the respective measurement regions of said at least two projection data acquisition systems.

17. A tomography method as claimed in claim 10 comprising, in an image reconstruction computer, reconstructing an image of said examination region from the projections acquired by said at least two acquisition systems.

18. A tomography method as claimed in claim 10 comprising mounting said at least two projection data acquisition systems in a computed tomography scanner for rotation around said examination region and, an image reconstruction computer, implementing a computed tomography image reconstruction algorithm using the projections acquired by said at least two projection data acquisition systems, to generate a computed tomography image of said examination region.
